(19) United States
(12) Patent Application Publication
(10) Pub. No.: US 2017/0199188 A1
(43) Pub. Date: Jul. 13, 2017

(54) METHOD OF PERFORMING ELECTROPOLYMERIZED ELECTROCHEMICALLY ACTIVE POLY-FILMS AS CURRENT SIGNAL TO DETECT BACTEREMIA

(71) Applicant: National Tsing Hua University, Hsinchu (TW)

(72) Inventors: Fan-Gang Tseng, Hsinchu (TW); Jen-Kuei Wu, Hsinchu (TW); Hwan-You Chang, Hsinchu (TW); Pen-Cheng Wang, Hsinchu (TW)

(21) Appl. No.: 15/163,051

(22) Filed: May 24, 2016

(30) Foreign Application Priority Data
Jan. 12, 2016 (TW) 105100794

(51) Int. Cl. G01N 33/569; G01N 27/327; B01L 3/00

(52) U.S. Cl. CPC G01N 33/56911; G01N 33/56938; B01L 3/502761; B01L 3/502738; B01L 3/502715; G01N 27/3277; B01L 2300/12; B01L 2300/0681; B01L 2200/0647; B01L 2300/0627

(57) ABSTRACT

The present invention is to provide a method of performing electropolymerized electrochemically active poly-films as a current signal to detect bacteremia. The method comprises conjugating an electrochemical redox-active molecular monomer and a specific antibody with a gold nanoparticle to form a modified gold nanoparticle, and the modified gold nanoparticles are conjugated to the surface of bacteria via a specific antibody to form an electrochemically active poly-film by electropolymerization. When applying a voltage, a redox-active current signal of the electropolymerized electrochemically active poly-films can be detected by a usual electrochemical detection system typically in the range between nA and mA.

### CROSS-REFERENCE TO RELATED APPLICATION

This application claims the priority of Taiwanese patent application No. 105100794, filed on Jan. 12, 2016, which is incorporated herewith by reference.

### BACKGROUND OF THE INVENTION

1. Field of the Invention

The present invention is to provide a method for the detection of bacteria, and more particularly, to provide a method of performing electropolymerized electrochemically active poly-films as current signal to detect bacteremia.

2. The Prior Arts

Bacterial invasion into the human circulatory system is referred to as bacteremia, often has serious consequences, including death. But bacteremia diagnosis currently relies on bacteria cultures, in which biochemical characterization can take several days to complete, and bacterial infection is still a threat to human health. Thus, developing a rapid, sensitive, and simple method for the detection of bacterial pathogens in whole blood could be highly urgent.

However, it is still a challenge to directly detect a few numbers of living bacteria (<5-10 cells/mL) in an industrial or clinical specimen. In conventional bacterial identification process, first observe individual colony morphology through a microscope after screening using selective media, and finally carry out a biochemical or serum test, which contains staining techniques, or metabolism and immune response for species-specific identification of strains. There is no method to identify all pathogens, so the clinical laboratory technologist should use different detection techniques to reduce the chance of false-positive results for some stains which are difficult to distinguish. Therefore, the professional technologist training and operating procedures are time-consuming to cause the problem.

Currently, an application integrated nanotechnology into a detection system has been gradually developed; the application of nanoparticles (NPs) can enhance the signal to noise ratio of the detection system. So far, a lot of biological detection systems integrated nanotechnologies for specific biomolecular detection (e.g. nucleic acids, proteins and enzymes) and quick detection for infectious diseases have been published. However, in electrochemical detection, there is a challenge about how to get enough signal to noise ratio given the detection system, usually a metal chelate complex is used to enhance transfer of the electrochemical signals. The method can detect the intensity of the fluorescence signal is necessarily limited in the amount of sample concentration (mg-ng/mL); it will be caused a problem of cross-interference or contamination by amplifying the fluorescence signal. In order to reach the sample concentration of a number of bacterial, it is more appropriate to use the method of electrochemical signals. In generally conventional electrochemical immunoassay, it provides a current signal based on specific enzyme reaction or an electrochemical signal based on redox functional groups on the sample surface. Therefore, the majority of electrochemical assay intends to improve sensitivity, in particular, focus on the development of the specific enzyme or increase the effect on enzyme-catalytic reaction (10⁵⁻⁸ cells/mL), and enhance the redox reaction through surface modification (10³⁻⁶ cells/mL). These methods are not been able to effectively reach the desired sensitivity of clinical detection (1-100 cell/mL).

### SUMMARY OF THE INVENTION

As such, the present invention is to provide a method of performing electropolymerized electrochemically active poly-films as a current signal to detect bacteremia. The method is to use an electrochemical redox-active molecular monomer conjugating with the gold nanoparticles to form a modified gold nanoparticles having a significant concentration of electrochemical redox-active molecular monomer, and the modified gold nanoparticles conjugated on the surface of bacteria via a specific antibody to form an electrochemically active poly-film by electropolymerization. When applying a voltage, a redox-active current signal of the electropolymerized electrochemically active poly-films can be detected by a usual electrochemical detection system typically in the range between nA and mA.

An objective of the present invention is to provide a method of performing electropolymerized electrochemically active poly-films as current signal to detect bacteremia, comprising: a. conjugating an electrochemical redox-active molecular monomer and a specific antibody with a gold nanoparticle to form a modified gold nanoparticle; b. incubating a sample with the modified gold nanoparticle that conjugates to a bacteria in the sample via the specific antibody; c. isolating the modified gold nanoparticle conjugated with the bacteria; and d. detecting a redox-active current signal of the modified gold nanoparticle conjugated with the bacteria by an electrochemical detection system in the range between nA and mA to identify whether the bacteria presents in the sample, wherein the modified gold nanoparticle is conjugated on the surface of the bacteria to form an electropolymerized electrochemically active poly-film.

Another objective of the present invention is to provide a modified gold nanoparticle for detecting a bacteria, comprising: an electrochemical redox-active molecular monomer; a specific antibody; and a gold nanoparticle, wherein the electrochemical redox-active molecular monomer and the specific antibody respectively conjugates on the surface thereof, wherein the modified gold for detecting a bacteria is conjugated on the surface of the bacteria to form an electropolymerized electrochemically active poly-film.

A further objective of the present invention is to provide a method of detecting a bacteria comprising: a. providing a microfluidic chip device and pushing the above nanoparticle and a sample into the microfluidic chip device, wherein the microfluidic chip device comprises a main channel and a filter valve comprising an array of multi-walled carbon nanotubes; b. isolating the nanoparticle conjugated with the bacteria in the sample by the filter valve; and c. detecting a redox-active current signal of the nanoparticle conjugated with the bacteria by an electrochemical detection system in the range between nA and mA to identify whether the bacteria presents in the sample.

According to an embodiment of the present invention, the electrochemical redox-active molecular monomer is 5-amino-2-mercapto-1,3,4-thiadiazole (AMT), 4-aminothiophenol (4-ATP), 2-aminothiophenol (2-ATP), 3-aminothiophenol (3-ATP), 2,2′-dithiodianiline, 4,4′-dithiodianiline, aniline, thiophene or pyrrole.

According to an embodiment of the present invention, a microfluidic chip device is used to isolate the modified gold nanoparticle conjugated with the bacteria in the step c, and the microfluidic chip device comprises a main channel and a filter valve comprising multi-walled carbon nanotubes.

According to an embodiment of the present invention, a detection sensitivity of the method is at least 10 bacteria/mL of the sample to conjugate with the modified gold nanoparticle.

According to an embodiment of the present invention, the gold nanoparticle is a gold-based nanoparticle or gold-coated silica oxide nanoparticle.

Accordingly, the method of the present invention is directly to increase the redox-active current signal (the electrochemical redox-active molecular monomer) on the surface (cell membrane of bacteria) of the analyte (bacteria), therefore, even if only a little bit bacteria also can be detected because there is sufficient amount of the electrochemical redox-active molecular monomer on the surface of the bacteria. When applying a voltage, a redox-active current signal of the electropolymerized electrochemically active poly-films can be detected by a usual electrochemical detection system typically in the range between nA and mA. Therefore, it is not necessary to combine with other technology and provide a highly sensitive electrochemical detection system (<PA) for an extremely low concentration measurement (pM-fM). The method of the present invention can significantly reduce the detection costs and have the convenience of operation.

### BRIEF DESCRIPTION OF THE DRAWINGS

The present invention will be apparent to those skilled in the art by reading the following detailed description of a preferred embodiment thereof, with reference to the attached drawings, in which:

FIG. 1 shows the method of the present invention to form an electropolymerized electrochemically active poly-film as current signal to detect bacteremia. P-AMT is polymerized 5-amino-2-mercapto-1,3,4-thiadiazole, P-4-AMT is polymerized 4-aminothiophenol;

FIG. 2 shows the structure of the microfluidic chip device in the present invention;

FIG. 3A shows cyclic voltammogram (CV) of the gold nanoparticles modified with 5-amino-2-mercapto-1,3,4-thiadiazole (AMT) electropolymerized on the surface of ITO electrodes.

FIG. 3B shows cyclic voltammogram (CV) of the gold nanoparticles modified with 4-aminothiophenol (4-ATP) electropolymerized on the surface of ITO electrodes;

FIG. 4A shows the specificity of the method of the present invention. Staphylococcus aureus (SA) is detected under the following conditions: (I) a mixture solution of the modified gold nanoparticles (RA-GNPs-Ab) (10³⁻⁴ particles/μL) and bacteria (SA) (dash line); (II) pure bacteria without RA-GNPs-Ab (gray line); and (III) control group: only RA-GNPs-Ab (solid line); and (a) the modified gold nanoparticles (RA-GNP-Ab) with bacteria matching target, and no response is observed (b) RA-GNPs-Ab with target mismatch; (c) RA-GNPs without antibody-target bacteria (fail to conjugate); and (d) pure target bacteria (from 0 up to 10⁵ CFU/mL);

FIG. 4B shows the specificity of the method of the present invention, Pseudomonas aeruginosa (PA) is detected under the following conditions: (I) a mixture solution of the modified gold nanoparticles (RA-GNPs-Ab) (10³⁻⁴ particles/μL) and bacteria (PA) (dash line); (II) pure bacteria without RA-GNPs-Ab (gray line); and (III) control group: only RA-GNPs-Ab (solid line); and (a) the modified gold nanoparticles (RA-GNP-Ab) with bacteria matching target, and no response is observed (b) RA-GNPs-Ab with target mismatch; (c) RA-GNPs without antibody-target bacteria (fail to conjugate); and (d) pure target bacteria (from 0 up to 10⁵ CFU/mL);

FIG. 5 shows the sensitivity of the method of the present invention, (I) E=0.8 V, a gold nanoparticle with P-AMT (P-AMT-GNPs) in Pseudomonas aeruginosa (PA) concentration (0.1-1.0 cells/μL) adds up to the volume of 10, 20, 30, 40, 50, 60, 70 μL (a→g); (II) the bacteria in blood plasma samples fail to conjugate with P-AMT-GNPs under the same conditions in (I); (III) E=1.0 V, the a gold nanoparticle with P-ATP (P-ATP-GNPs) in Staphylococcus aureus (SA) concentration (0.1-1.0 cells/μL) adds up to volume of 10, 20, 30, 40, 50, 60, 70 μL (a→g) (IV) the bacteria in blood plasma samples fail to conjugate with P-ATP-GNPs under the same conditions in (m).

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

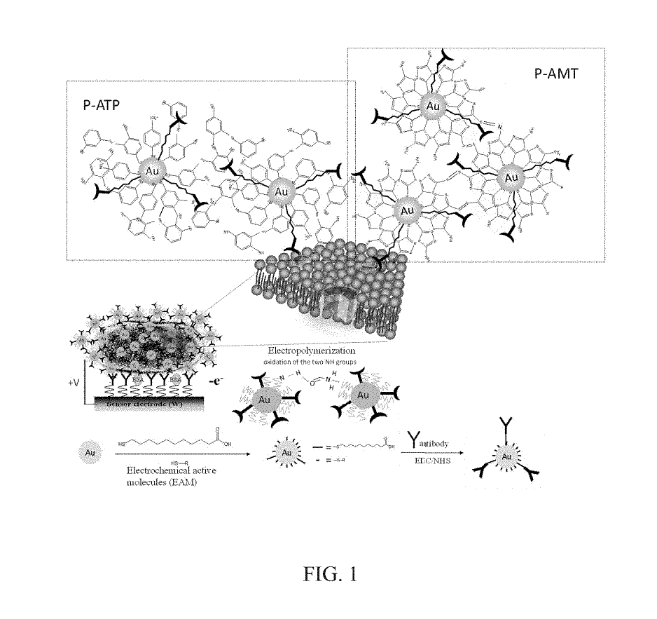

The present invention is to provide a method of performing electropolymerized electrochemically active poly-films as a current signal to detect bacteremia. The method is a detection technology applied in a microfluidic chip device; it is a novel pathogen identification system from whole blood. As shown in FIG. 1, the method of the present invention is to use an electrochemical redox-active molecular monomer, and the electrochemical redox-active molecular monomer can be 5-amino-2-mercapto-1,3,4-thiadiazole (AMT) or 4-aminothiophenol (4-ATP) to conjugate with the gold of gold nanoparticles forming the modified gold nanoparticles having a significant concentration of electrochemical redox-active molecular monomer, and the modified gold nanoparticles conjugate to the surface of bacteria via a specific antibody to form an electrochemically active poly-film by electropolymerization. The modified gold nanoparticles conjugating to the bacteria can be separated by a microfluidic chip device. When applying a voltage, a redox-active current signal of the electropolymerized electrochemically active poly-films can be detected by a usual electrochemical detection system typically in the range between nA and mA.

### Definition

As used herein, an electrochemical redox-active molecular monomer can be 5-amino-2-mercapto-1,3,4-thiadiazole (AMT), 4-aminothiophenol (4-ATP), 2-aminothiophenol (2-ATP), 3-aminothiophenol (3-ATP), 2,2′-dithiodianiline, 4,4′-dithiodianiline, aniline, thiophene or pyrrole.

As used herein, an electrochemical detection system typically in the range between nA and mA can be Bioanalytical Systems, Inc. (West Lafayette, Ind.) CHI 700 & 600 Series (e.g. CHI 760B) bipotential electrochemical workstations, BAS Inc. (USA & JAPAN) CS-3A Cell Stand and Pine Research Instrumentation AFCBP1 Bipotentiostat.

### Example 1

### Experimental Section

### 1.1 Chemicals and Cell Culture

All reagents used in the present invention are of analytical grade and the solutions are prepared using deionized water. Prior to each experiment, all solutions are filtered using 0.22 μm syringe filters (Whatman, NJ, USA). In one embodiment, the electrochemical redox-active molecular monomer is, for example but not limited to, 5-amino-2-mercapto-1,3,4-thiadiazole (AMT) or 4-aminothiophenol (4-ATP), which are purchased from Sigma-Aldrich (MO, USA).

In the present invention, 10 mM 4-(2-hydroxyethyl)-piperazine-1-ethanesulphonic acid (HEPES, Sigma, MO, USA) is prepared as a running buffer and adjusted to pH 7.5 using 0.1 N NaOH.

In one embodiment of the present invention can simultaneously detect two pathogenic, for example but not limited to, Staphylococcus aureus (SA) and Pseudomonas aeruginosa (PA), which are obtained from Bioresource Collection and Research Center (BCRC), Taiwan. The bacteria are grown in a culturing dish with 3 mL Luria-Bertani medium (BD Difco™, Cat. No 244610) supplemented with 10% heat-inactivated equine serum, 5% fetal bovine serum (Hyclone Laboratories, Logan, Utah), and 1% penicillin streptomycin solution (Sigma Chemical, MO, USA). All bacteria are cultured at 37° C. overnight (16 to 18 hr) under shaking at 150 rpm.

### 1.2 Chip Design and Fabrication

The microfluidic chip device used in the present invention is composed of a Pyrex glass substrate and a silicon wafer, which are an electrode chip and a fluidic chip. The microfluidic chip includes an incubation chamber connected to a loading channel via a filter valve comprising an array of multi-walled carbon nanotubes (MWCNTs), the size of the loading channel for loading sample is 1 cm in length, 100 μm in width and 10 μm in depth, and the size of the incubation chamber is 200 μm in length, 100 μm in width and 10 μm in depth, and the size of the filter valve from 5 to 10 μm in width, and 100 μm in length. The detection channel is 2 cm in length, 25 μm in width and 5 μm in depth. The construction of the microchannel is performed by soft-lithography technology. The silicon wafer is first coated with a silicon nitride layer by plasma enhanced chemical vapor deposition (PECVD) process, then coated with a thick film of AZ9620 photoresist by a photoresist spin coater. The microchannel patterns are transferred on photoresist by twice lithography steps, and produced a channel from about 5 and 10 μm in depth by reactive ion etching (1000E RIE system, Branchy. Taiwan). Layers of Ti, Al, and Ni metal catalysts (thicknesses of 1500, 200, and 100 Å, respectively) are consecutively deposited by e-beam evaporation on the surface patterned by photoresist for the growth of MWCNTs with layers for adhesion, electron conduction, and catalysis. Following the removal of the photoresist by a lift-off process, the vertical growth of MWCNTs in silicon channel is performed by thermal chemical vapour deposition (CVD). In the electrode chip, microelectrode location on Pyrex chip are defined by lithograph techniques, then 30 nm of titanium and 120 nm of platinum metal are deposited on a Pyrex chip to act as the adhesive layer and electrochemical electrode, respectively (platinum electrodes are as reference and counter electrodes). Working electrodes are patterned by using DC sputtering to deposit Indium Tin oxide (ITO), followed by using a lift-off process. After completing two chips, the chips are treated with O₂ plasma and corona to enhance bonding force prior to bonding. The flow direction and flow control of the microfluidic chip are controlled by a programmable pump (KDS 200P) and Push-Pull Syringe Pump (KDs 120), respectively, and fluid flow within the channel is monitored by a microscope with CCD camera (Coolsnap-cf, Roper Scientific GmbH, Germany).

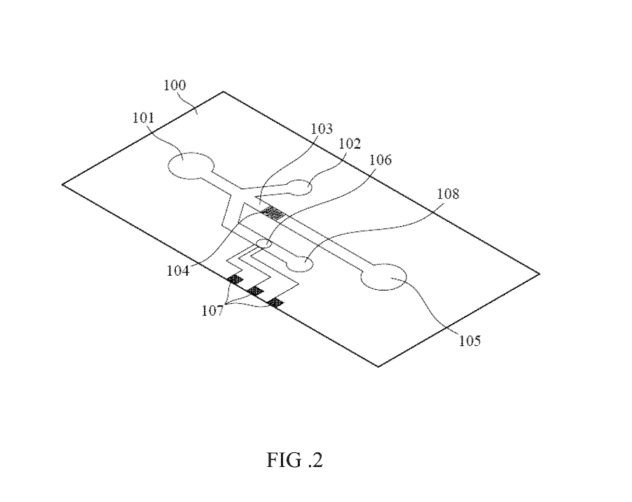

In the present invention, the microfluidic chip device 100 is composed of a main channel and a side channel, wherein the main channel is for a blood flow, and the side channel is for delivering the blood containing the modified gold nanoparticles to the position of detecting. As shown in FIG. 2, loading the whole blood sample into the loading channel 101 after centrifuge, simultaneously entering the modified gold nanoparticles in gold nanoparticles channel 102 and the whole blood sample into the incubation chamber 103 by controlling the microfluidic system, the end of incubation chamber 103 is connected to a filter valve 104 to filter the modified gold nanoparticles without conjugating with the surface antigens on bacteria and push them into the gold nanoparticles waste channel 105. The inter spacing between of the multi-walled carbon nanotubes in the filter valve 104 is about 100 nm to allow the entrance of the modified gold nanoparticles without conjugating with the surface antigens on the cell membrane of bacteria because the size of bacteria is about 1 to 3 μm and gold nanoparticles is about 20 to 60 nm. Then the purified modified gold nanoparticles having specific antibody conjugating with the cell membrane of bacteria push into a centralized channel with 20 μm in width and less than 5 μm in depth, which ensures that the modified gold nanoparticles conjugating with bacteria sequentially deliver to the downstream end before entering the detection channel 106. The modified gold nanoparticles conjugating with bacteria in the detection channel 106 can detect redox current signal using an electrochemical detection system 107, then them enter into the sample waste channel 108.

A spiked sample that blood plasma contaminated with Pseudomonas aeruginosa (PA) and Staphylococcus aureus (SA) is diluted in HEPES buffer at a ratio of 1:10. The dilution is calibrated for all stepwise concentration experiments. The electrochemical redox-active molecular monomer used in the present invention is 5-amino-2-mercapto-1,3,4-thiadiazole (AMT), 4-aminothiophenol (4-ATP), 2-aminothiophenol (2-ATP), 3-aminothiophenol (3-ATP), 2,2′-dithiodianiline, 4,4′-dithiodianiline, aniline, thiophene or pyrrole. In one embodiment, the modified gold nanoparticles having a significant concentration of electrochemical redox-active molecular monomer (RA-GNP-Ab) are formed by respectively conjugating AMT and 4-ATP with gold nanoparticles (GNPs) through the covalent bonds formed between thiol group and gold, and subsequently conjugating the bacterial specific antibodies with gold nanoparticles (GNPs) via the activation of 11-mercaptoundecanoic acid (2 mM) with 1-(3-Dimethylaminopropyl)-3-ethylcarbodiimide hydrochloride (EDC) and sulfo-NHS. The modified gold nanoparticles having a significant concentration of electrochemical active molecular (redox-active monomer) conjugate to the surface of bacteria via a specific antibody to form an electrochemically active poly-film by electropolymerization. The microfluidic chip having a filter valve undergoes consecutive filtering and washing with PBS buffer to obtain the GNPs-bacteria conjugates and to remove non-conjugated GNPs. Further elution of the retained GNPs-bacteria conjugates is accomplished by passing 5-10 mL of PBS in the direction opposite to that of the initial filtering, and injecting the outgoing solution of the eluted GNPs-bacteria conjugates in PBS directly into the measurement chamber to facilitate electrochemical detection using a bipotential electrochemical workstation (CHI 760B, Bioanalytical Systems, Inc., West Lafayette, Ind.).

Specially, the present invention is a method of performing electropolymerized electrochemically active poly-films as current signal to detect bacteremia, wherein the surface of gold nanoparticles can be modified with different electrochemical redox-active molecular monomers and specific antibodies. The different electrochemical redox-active molecular monomer can be applied in different bacteria detection due to the different current performance. Therefore, the method of the present invention can detect many bacterial pathogens at the same time.

Also, the overall reaction time of the method of performing electropolymerized electrochemically active poly-films as current signal to detect bacteremia can be completed within 30 min, the process is as follows: (1) preparing the modified gold nanoparticles (RA-GNP-Ab) and sample contaminated with bacteria, which spends 2 to 3 min; (2) filtering the GNPs-bacteria conjugates, which spends less than 10 min; (3) incubating the GNPs-bacteria conjugates in the incubation chamber and washing them with PBS buffer, which spends 3 to 5 min; (4) delivering the GNPs-bacteria conjugates into the measurement chamber for detection, which spends 3 to 5 min. Overall, there are various advantages of the method of the present invention, comprising rapid, sensitive, easy operation and portable, and it is for the use of clinical detection and rapid infectious disease screening.

### Example 2

### Characteristics of the Electrochemical Redox-Active Molecular Monomer in the Modified Gold Nanoparticles (RA-GNP-Ab)

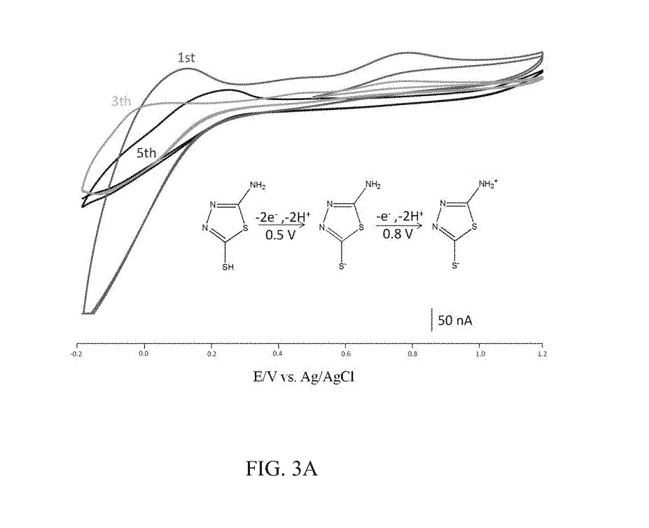

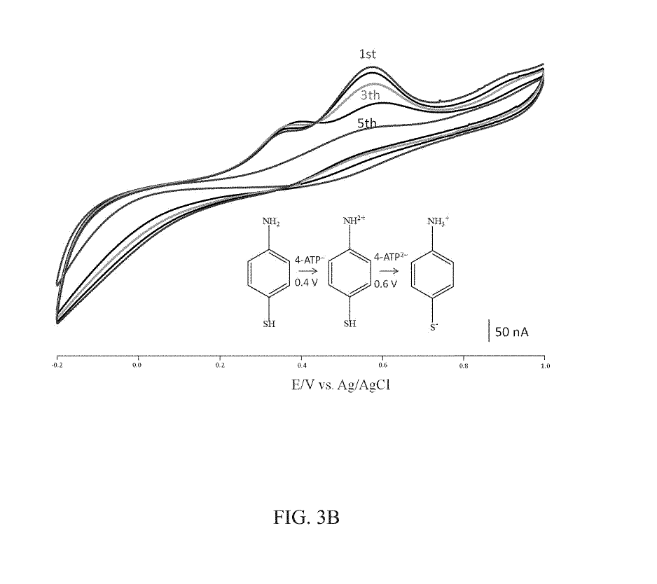

In the method of the present invention, the electropolymerized electrochemically active poly-film is formed by the covalent bonds between thiol group of the electrochemical redox-active molecular monomer, 5-amino-2-mercapto-1,3,4-thiadiazole (AMT) or 4-aminothiophenol (4-ATP) and gold nanoparticles (GNPs). The electrochemical properties of this monolayer are investigated using continuous cyclic voltammetry (CV) (5 cycles). FIG. 3A presents a cyclic voltammogram (CV) of the modified gold nanoparticles with AMT electropolymerized on the surface of ITO electrodes. The two cation radicals on the AMT are coupled to form a dimer via a hydrazone-bond after a voltage is applied. The oxidation of the two NH groups on the AMT leads to the formation of dimer species on the surface of the electrode. The oxidation of SH occurs in the voltage between 0.2 V and 0.6 V; therefore, it can be expected that the observed redox peak is due to a redox reaction of thiol to disulfide. In FIG. 3A, there are three oxidation peaks at 0.2 V, 0.5 V and 0.8 V in the forward scan and one reduction peak at −0.1 V in the reverse scan. At larger positive electrode potentials (over 1.0 V), an anodic wave is observed, which is presumably due to the desorption of polymerized AMT (P-AMT)-GNPs from the surface and concomitant oxidation reaction.

FIG. 3B presents cyclic voltammogram (CV) of the gold nanoparticles modified with 4-ATP electropolymerized on the surface of ITO electrodes. The anodic peak observed in the first cycle can be assigned to the electrochemical oxidation of the oxidizable mercapto group and pyrrole nitrogen of the monomer. An electrochemical polymerization process is used to form a polymerized-ATP gold nanoparticle (P-ATP GNPs) film on ITO electrodes. A film of P-ATP GNPs is deposited by repetitively sweeping the potential from −0.2 to 1.0 V at a scanning rate of 100 mVs⁻¹. An irreversible oxidation process appears during the first cycle and disappears during the second cycle. The reduction peak at 0.33 V may have been caused by the catalyzing of P-ATP polymerization by GNPs. An oxidation peak of P-ATP is clearly observed at potentials of 0.38 V and 0.58 V in the first scan. These results demonstrate the formation and bonding of a compact polymeric film to the surface of the electrode. The decrease in peak current appears to be related to the continual formation of P-ATP GNPs composite membranes leading to the suppression of the voltammetry response. The current gradually decreases with the number of scan cycles, eventually reaching a steady state.

As shown in FIGS. 3A and 3B, the electrochemical redox-active molecular monomers have different current performance; therefore, the method of the present invention can be applied to detecting different bacterial pathogen even at the same time.

### Example 3

### Specificity of the Method of the Present Invention

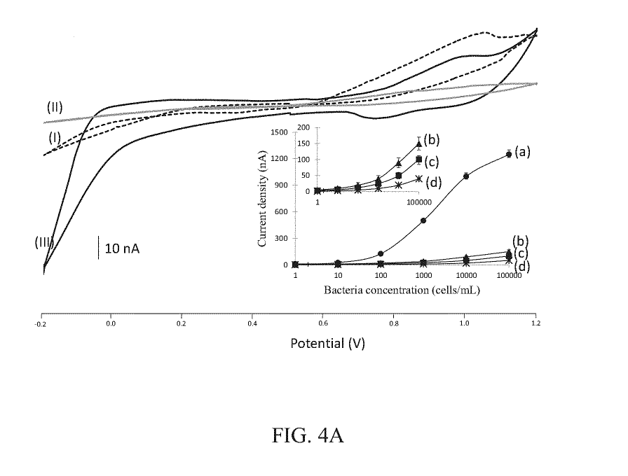

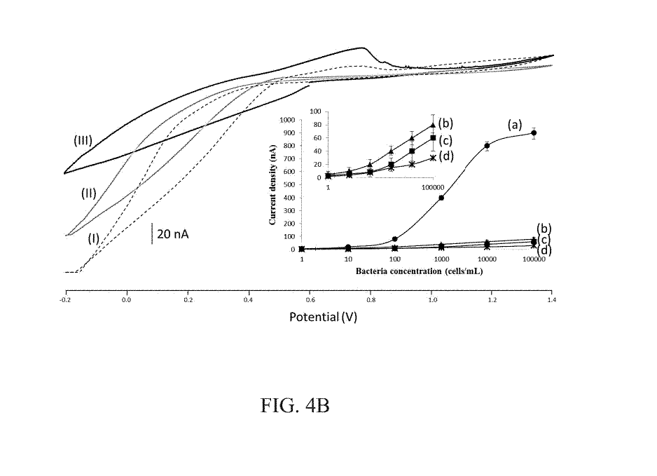

The specificity of the method of the present invention is evaluated by exposing the antibody-immobilized multi-array electrodes to Staphylococcus aureus (SA) and Pseudomonas aeruginosa (PA) under the following conditions: (I) a mixture solution of the modified gold nanoparticles (RA-GNPs-Ab) (10³⁻⁴ particles/μL) and bacteria (SA or PA); (II) pure bacteria without RA-GNPs-Ab; and (III) control group: only RA-GNPs-Ab. The bacteria sample is diluted to 10 cells/mL using 100 mM HEPES and 20 mM phosphate buffer. All of the CVs are detected for a potential range from −0.2 to +1.2 V vs. counter/reference electrode. All measurements are obtained at a scanning rate of 100 mV/s (total test volume is 100 μL). FIG. 4A shows the result of Staphylococcus aureus (SA), and FIG. 4B shows the result of Pseudomonas aeruginosa (PA). The cyclic voltammograms (CV) illustrates the signal enhancement under three conditions detected by the method of the present invention and signal enhancement, which verify whether the potentiometric signal is obtained exclusively from the bacterial recognition event of the biosensor rather than from the non-specific absorption of target bacteria or the leakage of residual RA-GNPs-Ab during the filtration step.

To verify the non-specific interaction in the method of the present invention, the modified gold nanoparticles are exposed to target bacteria with stepwise increases in concentration. Serum testing for the identification of bacteria is conducted using actual samples, FIG. 4A shows the result of Staphylococcus aureus (SA), and FIG. 4B shows the result of Pseudomonas aeruginosa (PA): (a) the modified gold nanoparticles (RA-GNP-Ab) with bacteria matching target, and no response is observed (b) RA-GNPs-Ab with target mismatch; (c) RA-GNPs without antibody-target bacteria (fail to conjugate); and (d) pure target bacteria (from 0 up to 10⁵ CFU/mL). These results demonstrate that no cross-reactivity occurred between these two pathogenic bacteria, and indicate the high specificity for this specific antibody.

### Example 4

### Sensitivity of the Method of the Present Invention

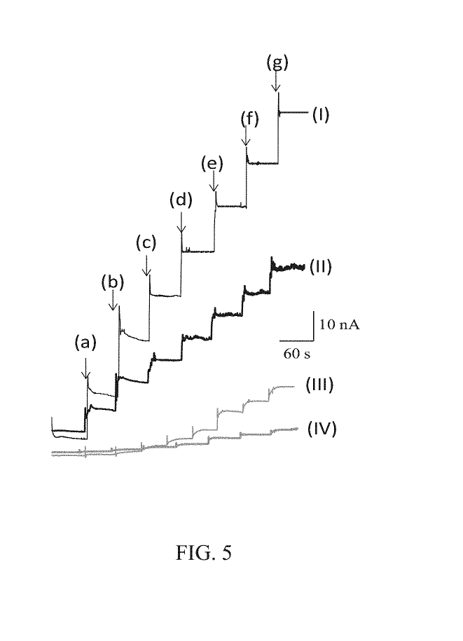

In the present invention, bacteria (SA and PA) at certain concentrations are diluted to 0.1-1.0 cells/μL using blood plasma and IX PBS buffer. Peaks are measured in the electrochemical current at 0.6 to 1.0 V for P-AMT and at 0.6 to 0.8 V for P-ATP resulting from cation-radical interactions. Scanning electron microscope (SEM) images are analysed using ImageJ software to calculate that single cells can be obtained from approximately 150-200 nanoparticles. As shown in FIG. 5, the sensitivity of detection for Pseudomonas aeruginosa (PA) is about 10 to 100 cells/mL, which is higher than that of Staphylococcus aureus (SA). This is probably due to the larger size of PA compared to SA, resulting in the production of a significantly stronger electrochemical signal by increasing the number of the modified gold nanoparticles conjugating on the surface of the bacteria. These results demonstrate that the method of performing electropolymerized electrochemically active poly-films as current signal to detect bacteremia has a very high sensitivity.

### Example 5

### Purification and Isolation of Bacteria in the Microfluidic Channel

The method of the present invention has the advantages of detection sensitivity and simplified procedure. Bacteria, the modified gold nanoparticles and the modified gold nanoparticles conjugated with bacteria are isolated by a filter valve comprising an array of multi-walled carbon nanotubes, the modified gold nanoparticles without conjugating with bacteria can be pushed into the filter valve by washing with PBS buffer, and the modified gold nanoparticles conjugated with bacteria can be pushed into the measurement chamber with a narrow microfluidic channel. The filter valve comprising an array of multi-walled carbon nanotubes has two functions: first, it can centralize the bacteria in the incubation chamber to increase antibody recognition and to detect a small number of bacteria in the sample; second, the filter valve can isolate the modified gold nanoparticles without conjugating with bacteria, which simplifies the step of purification.

After isolation, it needs to detect the modified gold nanoparticles A for identifying whether the bacteria presents in the sample using a usual electrochemical system typically in the range between nA and mA due to only a little bit bacteria (<100 cells/mL) in the sample.

The present invention is to provide a method of performing electropolymerized electrochemically active poly-films as a current signal to detect bacteremia. The purpose of the method is to detect bacteremia, the present invention has validated that the sensitivity of detection for two bacteria, Pseudomonas aeruginosa (PA) and Staphylococcus aureus (SA), can reach about 10 to 100 cells/mL in the blood by using the detection method with a isolating or concentrating device. Therefore, the present invention provides a high sensitive method to detect bacteremia.

In the present invention, the method is a detection technology applied in a microfluidic chip device; it is a high sensitivity pathogen identification system from whole blood. The method of the present invention is directly to increase the redox-active current signal by forming electrochemically active poly-films on the surface (cell membrane of bacteria) of the analyte (bacteria), the modified gold nanoparticles having a significant concentration of electrochemical redox-active molecular monomer can be conjugated with the surface of bacteria by specific antibody so as to detect a little bit bacteria within 30 min. When applying a voltage, a redox-active current signal of the electropolymerized electrochemically active poly-films can be detected by a usual electrochemical detection system typically in the range between nA and mA because there is sufficient amount of the electrochemical redox-active molecular monomer on the surface of the bacteria. Therefore, it is not necessary to combine with other technology and provide a highly sensitive electrochemical detection system (<PA) for an extremely low concentration measurement (pM-fM). The method of the present invention can significantly reduce the detection costs and have the convenience of operation.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A method of performing electropolymerized electrochemically active poly-films as current signal to detect bacteremia, comprising:
a. conjugating an electrochemical redox-active molecular monomer and a specific antibody with a gold nanoparticle to form a modified gold nanoparticle;
b. incubating a sample with the modified gold nanoparticle that conjugates to a bacteria in the sample via the specific antibody,
c. isolating the modified gold nanoparticle conjugated with the bacteria; and
d. detecting a redox-active current signal of the modified gold nanoparticle conjugated with the bacteria by an electrochemical detection system in the range between nA and mA to identify whether the bacteria presents in the sample,
wherein the modified gold nanoparticle is conjugated on the surface of the bacteria to form an electropolymerized electrochemically active poly-film.

2. The method according to claim 1, wherein the electrochemical redox-active molecular monomer is 5-amino-2-mercapto-1,3,4-thiadiazole (AMT), 4-aminothiophenol (4-ATP), 2-aminothiophenol (2-ATP), 3-aminothiophenol (3-ATP), 2,2′-dithiodianiline, 4,4′-dithiodianiline, aniline, thiophene or pyrrole.

3. The method according to claim 1, wherein a microfluidic chip device is used to isolate the modified gold nanoparticle conjugated with the bacteria in the step c, and the microfluidic chip device comprises a main channel and a filter valve comprising multi-walled carbon nanotubes.

4. The method according to claim 1, wherein a detection sensitivity of the method is at least 10 bacteria/mL of the sample to conjugate with the modified gold nanoparticle.

5. The method according to claim 1, wherein the gold nanoparticle is a gold-based nanoparticle or gold-coated silica oxide nanoparticle.

6. A modified gold nanoparticle for detecting a bacteria, comprising:
an electrochemical redox-active molecular monomer;
a specific antibody, and
a gold nanoparticle, wherein the electrochemical redox-active molecular monomer and the specific antibody respectively conjugates on the surface thereof,
wherein the modified gold nanoparticle for detecting a bacteria is conjugated on the surface of the bacteria to form an electropolymerized electrochemically active poly-film.

7. The modified gold nanoparticle according to claim 6, wherein the electrochemical redox-active molecular monomer is 5-amino-2-mercapto-1,3,4-thiadiazole (AMT), 4-aminothiophenol (4-ATP), 2-aminothiophenol (2-ATP), 3-aminothiophenol (3-ATP), 2,2′-dithiodianiline, 4,4′-dithiodianiline, aniline, thiophene or pyrrole.

8. The modified gold nanoparticle according to claim 6, wherein the gold nanoparticle is a gold-based nanoparticle or gold-coated silica oxide nanoparticle.

9. A method of detecting a bacteria, comprising:
a. providing a microfluidic chip device and pushing the nanoparticle according to claim 6 and a sample into the microfluidic chip device, wherein the microfluidic chip device comprises a main channel and a filter valve comprising an array of multi-walled carbon nanotubes;
b. isolating the nanoparticle conjugated with the bacteria in the sample by the filter valve; and
c. detecting a redox-active current signal of the nanoparticle conjugated with the bacteria by an electrochemical detection system in the range between nA and mA to identify whether the bacteria presents in the sample.

10. The method according to claim 9, wherein a detection sensitivity of the method is at least 10 bacteria/mL of the sample to conjugate with the modified gold nanoparticle.
